(19) United States
(12) Patent Application Publication
(10) Pub. No.: US 2016/0136332 A1
(43) Pub. Date: May 19, 2016

(54) THREE-DIMENSIONAL POROUS STRUCTURE MADE OF NANOFIBRE WEB FRAGMENTS AND METHODS FOR PRODUCTION THEREOF

(71) Applicants: FRAUNHOFER-GESELLSCHAFT ZUR FÖRDERUNG DER ANGEWANDTEN FORSCHUNG E.V., Müchen (DE); SPINPLANT GMBH, Leipzig (DE)

(72) Inventors: Sven HENNING, Halle (Saale) (DE); Andreas HEILMANN, Erdmannsdorf/Augustusburg (DE); Stefan SCHWAN, Halle (Saale) (DE); Andrea FRIEDMANN, Groitzsch (DE); Hans Joerg MEISEL, Berlin (DE); Timothy GANEY, Tampa, FL (US); Christian HERBST, Berlin (DE); Georg HILLRICHS, Goettingen (DE)

(73) Assignee: SPINPLANT GMBH, Leipzig (DE)

(21) Appl. No.: 14/899,851

(22) PCT Filed: Jun. 12, 2014
(86) PCT No.: PCT/EP2014/062258

§ 371 Date: Dec. 18, 2015

(30) Foreign Application Priority Data
Jun. 28, 2013 (DE) 10 2013 212 699.9

(51) Int. Cl. A61L 27/56; C12N 11/14; A61L 27/20; A61L 27/54; A61L 27/18; A61L 27/24

(52) U.S. Cl. CPC A61L 27/56; A61L 27/18; A61L 27/24; A61L 27/20; A61L 27/54; C12N 11/14; A61L 2300/406; A61L 2300/414; A61L 2300/64

(57) ABSTRACT

A three-dimensional, porous structure made of fragments of a nanofibre web is provided. Furthermore, a method for the production of a three-dimensional, porous structure made of nanofibre web fragments is proposed. The three-dimensional, porous structure is used for example in medicine, preferably in regenerative medicine. Furthermore, the structure according to the invention made of fragments of a nanofibre web can be used for the treatment of tissue damage, for the immobilisation of biological cells, for the construction of biological tissue and as a biological filler in vitro and also in vivo.

### FIELD OF THE INVENTION

A three-dimensional, porous structure made of fragments of a nanofibre web is provided. Furthermore, a method for the production of a three-dimensional, porous structure made of nanofibre web fragments is proposed. The three-dimensional, porous structure is used for example in medicine, preferably in regenerative medicine. Furthermore, the structure according to the invention made of fragments of a nanofibre web can be used for the treatment of tissue damage, for the immobilisation of biological cells, for the construction of biological tissue and as a biological filler in vitro and also in vivo.

### BACKGROUND

Webs, mats or felts (nonwovens) made of nanofibres display exceptional properties, such as a large specific surface, adjustable porosity and the possibility of being equipped with therapeutically active substances which make such nanowebs of interest for applications in medicine (Kittelmann, W. “Vliesstoffe: Rohstoffe, Herstellung, Anwendung, Eigenschaften, Prüfung” (nonwovens: raw materials, production, application, properties, testing), Wiley-VCH, Weinheim, 2000; Zahedia, P. et al., Polym. Adv. Technol., 2010, Vol. 21, pages 77-95; Schofer, M. D. et al., PLoS One, 2011, Vol. 6, p. 9).

In the last few years, in particular the development and production of nanowebs with fibre diameters of a few 10 nanometres to a few micrometres by means of electrospinning have thereby come into focus. In the meantime, these webs made of electrospun nanofibres are manufactured commercially and used already to a small extent for various cases of use, e.g. as a component of implants.

Optimisation of the fibre properties has progressed widely, with respect to material selection, fibre diameter and fibre length, also stable active substance inclusions in the fibres and also incorporation of crosslinking agents have been achieved in the meantime. Presently available web materials are produced typically as thin mats.

Because of the novelty and fragility of these webs, sterilisation and subsequent surface modification of the web materials and the development of methods for further processing and production of the webs represent an unresolved technical problem which to date has hindered commercial use of such products. To date, methods for the production of semifinished products of a defined size and geometry have not been available. In particular, methods are sought for manufacturing fairly small portions with a few millimetres or a few 100 micrometres edge length from planar webs and for forming three-dimensional structures such as hollow spheres, tubes and/or rods therefrom. Such products are advantageous for cell colonisation by the body's own cells of the recipient tissue (e.g. chondrocytes) in vitro and subsequent application.

To date, in addition methods for application of such products have not been available, clinical and economic success requiring to be based on the usability of the new material and also the new material after colonisation with living cells for application by means of established surgical methods. Whilst covering surface defects, e.g. of the skin or bone, with planar web products is relatively successful, filling of cavities or reaching not readily accessible defects in bone or in cartilage have to date been precluded. Products which can be introduced into defects by injection or are filled by pressing a pasty substance into the defect and which remain in position are not available. The operative methods should thereby correspond, e.g. to the routines when introducing conventional bone cements of different viscosity (pasty to injectable).

The production of planar webs of different dimensions, i.e. in a size adapted to the defect to be covered, is achieved without problem. The shaping of directed structures by the use of structured templates or suitable control of the electrical field during the spinning process is possible. Such structures can in principle also be further processed to form simple three-dimensional constructs (e.g. tubes) (WO 2012/112564 A1, Agarwal, J. H. et al., Polymer, 2008, Vol. 49, pages 5603-5621; Buttafoco, L. et al., J. Control. Release, 2005, Vol. 101, pages 322-324; Yang, F. et al., Biomaterials, 2005, Vol. 26, pages 2603-2610; Lee, Y.-S. & Arinzeh, T. L., Polymers, 2011, Vol. 3, pages 413-426; Cui, W. et al., Sci., Technol. Adv. Mater., 2010, Vol. 11).

Use of self-organisation effects for forming and shaping microscopic objects from nanofibre webs or portions or fragments produced therefrom has to date not been successful. Methods for colonising web layers in a bioreactor with subsequent stacking of the colonised layers have been reported. By these means, a three-dimensional structure, colonised by cells, has to some extent been achieved. However, application of such products in cavities and not readily accessible defects remains problematic.

Successful cell colonisation of prescribed three-dimensional structures produced for example by layering of a plurality of web layers has been achieved only superficially since growth of cells in very small-pore structures (size <200 to 500 μm) is precluded and supplying cells in the interior of the constructs cannot be guaranteed.

### SUMMARY OF THE INVENTION

The object was hence the provision of a method for the production of a three-dimensional, porous structure made of fragments which consists of at least one web made of nanofibres and which is suitable for filling with small-pore structures, i.e. structures of a size less than 200 to 500 μm.

The object is achieved by one or more embodiments disclosed and/or described herein.

### DETAILED DESCRIPTION OF THE INVENTION

According to the invention, a method for the production of a three-dimensional, porous structure made of fragments which consist of a web made of nanofibres is provided, comprising
a) cutting a dry or wet web made of nanofibres into fragments with a laser and suspending the web fragments in a liquid medium; or
b) cutting a web made of nanofibres which is present in a liquid medium into fragments with a laser, as a result of which a suspension of web fragments in the liquid medium is produced; and
c) at least partial removal of the liquid medium, a three-dimensional, porous structure being formed from web fragments by means of self-organisation.

According to the invention, there is understood by the term “fragment”, a small piece or a small portion of a large web, there requiring to be understood by the term “web”, a strip of a specific width produced in a continuous process. A web can also be produced by a discontinuous process which is completed with the complete covering of a fixed base. In the sense of the invention, fragments of a web are hereby “cut out” from the web by means of laser, i.e. separated out of the web. These fragments or pieces or portions can only be produced in this way, have new, additional properties relative to compact web strips and form the basic element or the basic building block of the application described here.

According to the invention, there is understood by the term “nanofibres”, fibres which have a diameter of 1 nm to 10 μm.

By means of withdrawal of the liquid medium in step c), adhesion of the surfaces of adjacent web fragments is effected and hence formation and compaction of a three-dimensional, porous structure is achieved. Consequently, a mechanically stable structure is produced.

The dry or wet web can have been produced by electrospinning of micro- and nanofibres. This can thereby concern a microfibre web or nanofibre web.

Cutting the web can take place in a continuous or discontinuous (batch) process. Also a plurality of web layers can be laid one upon the other and be cut at the same time.

The laser can concern a laser in continuous operation or a laser with pulse durations in the nanosecond range or ultrashort pulse range. (picoseconds to femtoseconds). Typical wavelengths of the radiated light are situated in the range of 193 nm to 1,100 nm. However the laser can also be a continuously or pulsed-operating laser in the infrared range (i.e. λ in the IR spectrum) (e.g. a CO₂ laser). Preferably, the laser emits radiation of a wavelength of approx. 10 μm and an intensity ≧10 kW/cm². Further preferred is a holmium laser or an erbium laser.

The energy densities or fluences which are used when cutting dry or wet webs with pulsed lasers are preferably in the range of 0.1 J/cm² to 50 J/cm².

The average powers of the pulsed lasers which are possible are preferably ≧100 mW.

Typical cutting rates are between 1 mm/s and 1,000 mm/s, optionally 10 mm/s and 100 mm/s.

Formation of the three-dimensional, porous structure made of web fragments via self-organisation can be effected by non-covalent and/or covalent interactions. By means of specific surface modification of the web, implemented before steps a) or b) (e.g. activation, production of reactive functionalities) or the use of a chemically functionalised web in step a) or b), for example compaction of the three-dimensional, porous structure can be effected via (specific) covalent chemical bonds.

A crucial advantage of the method according to the invention is that, by the use of a laser, web fragments which would not be accessible via a mechanical cutting method can be produced. Known mechanical methods would not permit production at all or extensively damage the advantageous nanostructures. The production, which is possible due to the method according to the invention, of such sufficiently small web fragments without damage to the micro- and nanofibre structure thereof is a prerequisite for achieving the advantageous properties of the three-dimensional, porous structure according to the invention.

In the case of using a laser as cutting tool, the method according to the invention offers—in addition to the cutting function—also the possibility of specific surface modification and sterilisation of the product.

In the method according to the invention, a gel, a paste or a solid structure can be formed in step c), in particular a structure serving as biological extracellular matrix. This can be controlled by controlled withdrawal of the liquid medium in the method according to the invention, accumulation of the web fragments and/or web fragments and adhering cells being effected by withdrawal of the suspension medium. By the addition of biological cells in step a) or step b), a three-dimensional hybrid of web fragments and cells is hence produced.

The three-dimensional structure of the framework, in particular the porosity thereof, is prescribed by the size and shape of the web fragments.

By means of progressive withdrawal of the suspension medium, further compaction of the three-dimensional arrangement and adhesion of the substrate surfaces can be effected, as a result of which compaction of the hybrid system is achieved.

In step a) or b) of the method according to the invention, the web can be cut into polygonal web fragments, web fragments with rounded edges, round web fragments, triangular web fragments, square web fragments, rectangular web fragments, rhomboid web fragments and/or trapezoidal web fragments.

Furthermore, fragments with an edge length of 50 μm to 100 mm, optionally 100 μm to 10 mm, can be produced in step a) or step b). Preferably, fragments with a surface area of 1 mm² are produced.

After step a) or step b), the web fragments and/or, after step c), the porous three-dimensional structure can be contacted with biological cells, preferably with human cells, in particular with chondrocytes, osteoblasts, fibroblasts and/or stem cells, human embryonic stem cells being the exception. The individual fragments which are present spatially separated from each other in the liquid medium hereby serve as substrates for accumulation and proliferation of the cells.

The method according to the invention can be characterised in that, in step a), the web fragments are suspended in water, in a physiological common salt solution and/or in a nutrient medium for cell cultures or be present therein in step b). The advantage of a wet web in step a) or web in a liquid medium in step b), relative to a dry web in step a), is that the former can be suspended more easily and effectively.

The web can be irradiated with the laser, in step a), contained in a gaseous medium or consisting of air, inert gas and/or process gas. Furthermore, the web can be present, in step b), contained in a liquid medium or consisting of water, physiological common salt solution and/or nutrient medium for cell cultures.

Preferably, in step a) of the method, a web is used which comprises
i) nanofibres with a diameter of 10 nm to 10 μm, preferably with a diameter of 50 nm to 500 nm;
ii) nanofibres made of biocompatible, resorbable or non-resorbable, synthetic or natural polymers, preferably polymers selected from the group consisting of poly-L-lactide, poly-D-lactide, poly-(D,L)-lactide, poly-(L-lactide-co-D,L-lactide), polyglycolic acid, poly-(lactide-co-glycolide), polyhydroxybutyrate and poly-(hydroxybutyrate-co-hydroxyvalerate), and also mixtures hereof;
iii) nanofibres made of resorbable, biocompatible, natural polymers, preferably collagen, crosslinked collagen, chitosan or comparable materials;
iv) bioactive fillers, preferably hydroxyapatite and/or tricalcium phosphate, optionally α-tricalcium phosphate and/or β-tricalcium phosphate, and also mixtures hereof;
v) active substances, preferably antibiotics and/or growth factors; and/or
vi) additives, preferably colourants, particularly preferably fluorescent dyes, in particular chlorophyll.

Before step a) or step b) of the method according to the invention, the web made of nanofibres can be treated with a plasma, with a laser, preferably with a UV laser, or with UV radiation. In particular,
i) the web can hereby be sterilised;
ii) the physical and/or chemical properties of a surface of the web can be modified at least in regions, as a result of which in particular a hydrophilic and/or hydrophobic web is produced at least in regions;
iii) the physical and/or chemical properties of a web upper side and web underside can be modified at least in regions such that an amphiphilic web is produced and/or
iv) the web can be chemically functionalised on its surface, in particular by plasma polymerisation.

One advantage of the chemical hydrophobisation. hydrophilisation, amphiphilisation and/or modification with functional groups is the specific adaptation of the three-dimensional, porous structure of web fragments, produced via the method, to a specific target, i.e. for example when using the structure as filler, to the properties of a specific material used for filling. Furthermore, a three-dimensional, porous structure can hence be generated, which is reinforced or can be reinforced by covalent chemical bonds (within one web fragment and/or between different web fragments). In this respect, for example a thermal curing of the structure can be effected to form an intermolecular crosslinking of molecules of the same and/or different web fragments after the structure has been placed as filling material in a bone.

By specific adjustment or choice of the fragmentation in step a) or step b), by the choice of liquid medium in step a) or step b) and/or by the choice of a pretreatment of the web with plasma before step a) or step b), the web particles can assume specifically the following structure after being suspended:
i) an elongated, plate-shaped structure;
ii) a spherical structure, preferably micelles;
iii) a cylindrical structure, preferably microtubes; and/or
iv) mixtures or aggregates of these structures.

Furthermore, a three-dimensional, porous structure which comprises fragments of at least one web made of nanofibres or consists thereof is provided according to the invention, the fragments having an edge length of 50 μm to 100 mm and/or a surface area of ≦1 mm². Optionally, the edge length can be situated in the range of 100 μm to 10 mm.

The three-dimensional, porous structure can be characterised in that the fragments of the at least one web made of nanofibres have at least one cut edge at least partially and/or in regions, which was cut by laser radiation.

The three-dimensional, porous structure can receive or have biological cells, preferably human cells, in particular chondrocytes, osteoblasts, fibroblasts and/or stem cells, human embryonic stem cells being the exception. Preferably, these cells are bonded to at least one surface of the structure. The bonding can be effected via non-covalent and/or covalent chemical interactions, non-covalent interactions being preferred.

The three-dimensional, porous structure according to the invention can be producible or produced according to the method according to the invention. The three-dimensional porous structure can be suspended in a liquid medium.

Preferably, the three-dimensional, porous structure is used in medicine, in particular in regenerative medicine. Preferably, the three-dimensional, porous structure is applied by injection.

For particular preference, the three-dimensional, porous structure is used in the treatment of tissue damage, preferably bone damage, cartilage damage, intervertebral discs and/or skin damage.

Furthermore, the three-dimensional, porous structure can be used as substrate for the immobilisation of biological cells. Human cells, in particular chondrocytes, osteoblasts, fibroblasts and/or stem cells are hereby preferred, human embryonic stem cells being the exception.

Finally, the three-dimensional, porous structure is suitable for the construction of biological tissue and/or as biological filler, preferably as filler for bones, cartilage and/or skin.

Furthermore, it is proposed to use the porous structure according to the invention in vitro for one of the above-mentioned purposes.

The subject according to the invention is intended to be explained in more detail with reference to the subsequent examples without wishing to restrict said subject to the specific embodiments represented here.

### Example 1

### Production of Various Forms of the Three-Dimensional, Porous Structure Made of Web Fragments

The applicability of the three-dimensional structure according to the invention for cell colonisation and the use as product which can be implanted by injection are the shape, specific surfaces and size of the web particles. These key parameters are dependent, on the one hand, upon the chemical and physical properties of the web which is used and, on the other hand, upon the chosen process parameters during plasma treatment and the laser cutting of the web or of the web fragments.

By way of example before production of the micro- and nanofibre webs by laser, a plasma treatment for modifying the physical and chemical properties of the fibre surfaces or of the web surfaces or a surface functionalisation by plasma polymerisation is effected.

By the choice of shape, size and side length ratio of the web fragments and/or of the surface properties or surface functionalisation (e.g. hydrophilic, hydrophobic, amphiphilic) and also of the suspension medium and the concentration, the fragments in these suspensions can form various three-dimensional structures which are specifically adjustable.

In particular, planar (plates), cylindrical (rods and tubes) and spherical (balls and hollow balls) structures are producible.

For example, the following shape variations of the web particles (fragments) are adjustable: elongated, plate-shaped shape of the web fragments with good compatibility of web surface and suspension medium, spherical structures (micelles) by minimising the surface in the case of incompatibility of polymer surface and suspension medium, microtubes by rolling up the webs in the case of an amphiphilic surface configuration and use of rectangular fragments in the case of a high side length ratio, and also aggregation of the above-mentioned structures to form larger units.

### Example 2

### Structure of Collagen Fibre Web Fragments and Osteocytes or Chondrocytes and Use Thereof

Injectable three-dimensional, porous structures made of nanocollagen fibres and osteocytes are particularly suitable for filling not readily accessible bone defect zones. This can be necessary in the case of a reconstruction operation which is not one hundred per cent correctly adapted if small residual gaps are filled with this material. This is indicated in particular in the case of critical operations, e.g. such as operations in the septic field.

However also not readily accessible bone defects during reconstruction operations represent a medical indication especially when the bone production must be stimulated.

In the field of the cartilage, all osteoarthritic cartilage defects which are accessible by means of orthoscopic methods should be mentioned. Here a three-dimensional, porous structure made of collagen fibre web fragments and chondrocytes can be introduced into arthroscopically prepared cartilage defects. In particular defects in not readily accessible joints are thereby conceivable, such as e.g. hip-, shoulder- or ankle joint.

Of course, also the knee joint is a good target organ for such an injected cell-collagen composite. Here, the material according to the invention and the application form of the injection revealed by the properties of the material according to the invention offers an improvement with respect to previous methods in which so-called 3D constructs are introduced as cell-polymer hybrids.

What is claimed is:

1. A method for the production of a three-dimensional porous structure made of fragments which consist of a web made of nanofibres, comprising
a) cutting a dry or wet web made of nanofibres into fragments with a laser and suspending the web fragments in a liquid medium; or
b) cutting a web made of nanofibres which is present in a liquid medium into fragments with a laser, as a result of which a suspension of web fragments in the liquid medium is produced; and
c) at least partial removal of the liquid medium, a three-dimensional, porous structure being formed from web fragments by means of self-organisation.

2. The method according to claim 1, wherein in step c), a gel, a paste or a solid structure, in particular a structure serving as biological extracellular matrix is formed.

3. The method according to claim 1 wherein step a) or step b), the web is cut into polygonal web fragments, web fragments with rounded edges, round web fragments, triangular web fragments, square web fragments, rectangular web fragments, rhomboid web fragments and/or trapezoidal web fragments.

4. The method according to claim 1, wherein step a) or step b), fragments with an edge length of 50 μm to 100 mm and/or with a surface area ≦1 mm² are produced.

5. The method according to claim 1, wherein after step a) or step b), the web fragments and/or, after step c), the porous, three-dimensional structure is/are contacted with biological cells, preferably with human cells, in particular with chondrocytes, osteoblasts, fibroblasts and/or stem cells, with the exception of human embryonic stem cells.

6. The method according to claim 1, wherein the web, in step a), contained in a gaseous medium or consisting of air, inert gas and/or process gas, is irradiated with the laser, and/or the web, in step b), is present, contained in a liquid medium or consisting of water, physiological common salt solution and/or nutrient medium for cell culture.

7. The method according to claim 1, wherein in step a), the web is irradiated with the laser in a gaseous or liquid medium, preferably air, inert gas, process gas, water, physiological common salt solution and nutrient medium for cell cultures.

8. The method according to claim 1, wherein in step a), a web is used which comprises
i) nanofibres with a diameter of 10 nm to 10 μm, preferably with a diameter of 50 nm to 500 nm;
ii) nanofibres made of biocompatible, resorbable or non-resorbable, synthetic or natural polymers, preferably polymers selected from the group consisting of poly-L-lactide, poly-D-lactide, poly-(D,L)-lactide, poly-(L-lactide-co-D,L-lactide), polyglycolic acid, poly-(lactide-co-glycolide), polyhydroxybutyrate and poly-(hydroxybutyrate-co-hydroxyvalerate), and also mixtures hereof;
iii) nanofibres made of resorbable, biocompatible, natural polymers, preferably collagen, crosslinked chitosan or comparable materials, or
iv) bioactive fillers, preferably hydroxyapatite and/or tricalcium phosphate, optionally α-tricalcium phosphate and/or β-tricalcium phosphate, and also mixtures hereof;
v) active substances, preferably antibiotics and/or growth factors; and/or
vi) additives, preferably colourants, particularly preferably fluorescent dyes, in particular chlorophyll.

9. The method according to claim 1, wherein before step a) or step b), the web made of nanofibres is treated with a plasma, with a laser, preferably a UV laser, or with UV radiation, in particular
i) the web being sterilised;
ii) the physical and/or chemical properties of a surface of the web being modified at least in regions, as a result of which in particular a hydrophilic and/or hydrophobic web is produced at least in regions;
iii) the physical and/or chemical properties of a web upper side and web underside being modified at least in regions such that an amphiphilic web is produced, and/or
iv) the web being chemically functionalised on its surface, in particular by plasma polymerisation.

10. The method according to claim 1, wherein, by the choice of the fragmentation in step a) or step b), by the choice of the liquid medium in step a) or step b) and/or by the choice of a pretreatment of the web with plasma before step a) or b), the web particles assume specifically the following structure after being suspended:
i) an elongated, plate-shaped structure;
ii) a spherical structure, preferably micelles;
iii) a cylindrical structure, preferably microtubes; and/or
iv) mixtures or aggregates of these structures.

11. A three-dimensional, porous structure which comprises fragments of at least one web made of nanofibres or consists thereof, the fragments having an edge length of 50 μm to 100 mm and/or a surface area of ≦1 mm².

12. A three-dimensional, porous structure according to claim 11, wherein the fragments of the at least one web made of nanofibres have at least one cut edge, at least partially and/or in regions, which was cut by laser radiation.

13. The three-dimensional, porous structure according to claim 11, wherein the three-dimensional, porous structure has biological cells, preferably human cells, in particular chondrocytes, osteoblasts, fibroblasts and/or stem cells, with the exception of human embryonic stem cells, these cells being bonded preferably to at least one surface of the structure.

14. A three-dimensional, porous structure which comprises fragments of at least one web made of nanofibres or consists thereof, the fragments having an edge length of 50 μm to 100 mm and/or a surface area of ≦1 mm² made according to the method of claim 1.

15. The three-dimensional, porous structure according to claim 11 operable to be used in medicine, in particular in regenerative medicine.

16. The three-dimensional, porous structure according to claim 11 operable to be used
a) in the treatment of tissue damage, preferably bone damage, cartilage damage, intervertebral discs and/or skin damage;
b) in the immobilisation of biological cells, preferably human cells, in particular chondrocytes, osteoblasts, fibroblasts and/or stem cells, human embryonic stem cells being the exception; and/or
c) in the construction of biological tissue; and/or
d) as biological filler, preferably as filler for bones, cartilage and/or skin.

17. The three-dimensional, porous structure according claim 11, operable to be used in vitro
a) in the treatment of tissue damage, preferably bone damage, cartilage damage, intervertebral discs and/or skin damage;
b) for the immobilisation of biological cells, preferably human cells, in particular chondrocytes, osteoblasts, fibroblasts and/or stem cells, human embryonic stem cells being the exception; and/or
c) in the construction of biological tissue; and/or
d) as biological filler, preferably as filler for bones, cartilage and/or skin.

18. The method according to claim 4, wherein fragments with an edge length of 100 μm to 10 mm, and/or with a surface area ≦1 mm² are produced.

19. The method according to claim 5, wherein the biological cells are human cells selected from chondrocytes, osteoblasts, fibroblasts and/or stem cells, with the exception of human embryonic stem cells.

20. The three-dimensional, porous structure according to claim 13, wherein the biological cells are human cells selected from chondrocytes, osteoblasts, fibroblasts and/or stem cells, with the exception of human embryonic stem cells.
